(19) United States
(12) Patent Application Publication
(10) Pub. No.: US 2008/0262594 A1
(43) Pub. Date: Oct. 23, 2008

(54) Stent Graft Sealing System and Method

(75) Inventor: Jonathan Morris, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/737,327

(22) Filed: Apr. 19, 2007

(51) Int. Cl. A61F 2/06

(52) U.S. Cl. 623/1.13

(57) ABSTRACT

A stent graft sealing system and method including a stent graft having a seal stent, and a tubular graft material having a central axis and a pleated seal region. The pleated seal region is supported by the seal stent and urged outward from the central axis by the seal stent. The pleated seal region has an initial deployment diameter and an extended deployment diameter. The extended deployment diameter is greater than one and one-half times the initial deployment diameter.

### TECHNICAL FIELD

The technical field of this disclosure is medical implantation devices, particularly, a stent graft sealing system and method.

### BACKGROUND OF THE INVENTION

Wide ranges of medical treatments have been developed using endoluminal prostheses, which are medical devices adapted for temporary or permanent implantation within a body lumen, such as naturally occurring or artificially made lumens. Examples of body lumens in which endoluminal prostheses may be implanted include arteries such as those located within coronary, mesentery, peripheral, or cerebral vasculature; veins; gastrointestinal tract; biliary tract; urethra; trachea; hepatic shunts; and fallopian tubes. Various types of endoluminal prostheses have also been developed with particular structure to modify the mechanics of the targeted luminal wall.

A number of vascular devices have been developed for replacing, supplementing, or excluding portions of blood vessels. These vascular devices include endoluminal vascular prostheses and stent grafts. Aneurysm exclusion devices, such as abdominal aortic aneurysm (AAA) devices, are used to exclude vascular aneurysms and provide a prosthetic lumen for the flow of blood. Vascular aneurysms are the result of abnormal dilation of a blood vessel, usually from disease or a genetic predisposition, which can weaken the arterial wall and allow it to expand. Aneurysms can occur in any blood vessel, but most occur in the aorta and peripheral arteries, with the majority of aneurysms occurring in the abdominal aorta. An abdominal aortic aneurysm typically begins below the renal arteries and extends into one or both of the iliac arteries.

Aneurysms, especially abdominal aortic aneurysms, are commonly treated in open surgery procedures in which the diseased vessel segment is bypassed and repaired with an artificial vascular graft. While open surgery is an effective surgical technique in light of the risk of a fatal abdominal aortic aneurysm rupture, the open surgical technique suffers from a number of disadvantages. The surgical procedure is complex, requires a long hospital stay, requires a long recovery time, and has a high mortality rate. Less invasive devices and techniques have been developed to avoid these disadvantages. Tubular endoluminal prostheses that provide a lumen or lumens for blood flow while excluding blood flow to the aneurysm site are introduced into the blood vessel using a catheter in a less or minimally invasive technique. The tubular endoluminal prosthesis is introduced in a small diameter crimped condition and expanded at the aneurysm. Although often referred to as stent grafts, these tubular endoluminal prostheses differ from covered stents in that they are not used to mechanically prop open natural blood vessels. Rather, they are used to secure an artificial lumen in a sealing engagement with the vessel wall without further opening the abnormally dilated natural blood vessel.

Stent grafts for use in abdominal aortic aneurysms typically include a support structure supporting woven or interlocked graft material. Examples of woven graft materials are woven polymer materials, e.g., Dacron, or polytetrafluoroethylene (PTFE). Interlocked graft materials include knit, stretch, and velour materials. The graft material is secured to the inner or outer diameter of the support structure, which supports the graft material and/or holds it in place against a luminal wall. The stent graft is secured to a vessel wall above and below the aneurysm. A proximal spring stent of the stent graft can be located above the aneurysm to provide a radial force which engages the lumen wall and seals the stent graft at the lumen wall. The proximal spring stent can include hooks to puncture the vessel wall and further secure the stent graft in place.

Another use for stent grafts is treatment of traumatic aortic injuries, such as traumatic aortic transections. Traumatic aortic injuries often occur in the thoracic aortas of relatively young persons, a population which is prone to accidents.

One problem in present stent graft designs is sealing the proximal end of the stent graft to the vessel wall. Sealing the stent graft to the vessel wall prevents fluid from bypassing the stent graft lumen and flowing between the graft material and the vessel wall. The sealing problem is particularly acute in treatment of traumatic aortic injuries in young persons, whose aortas expand as they age. The radial expansion of present stent grafts is limited by the diameter of the graft material attached to the stent, typically being limited to ten to twenty percent of the original diameter of the stent graft as deployed in the vessel. This is often insufficient to maintain the seal for a growing aorta. When the stent graft diameter becomes too small, the stent graft will leak and may even migrate from the deployment site to an undesirable location. Although the aorta may have healed from the trauma, risky and expensive surgery may be required to remove the now undersized stent.

Another problem in present stent graft designs is sizing the stent graft for proper sealing. The graft material limits the range of diameters for which the stent graft can be used. The vessel diameter must be carefully determined and a stent graft covering a narrow diameter range selected. Errors in measurement can result in selection and deployment of a stent graft that is too small, resulting in seal leakage and a poor medical outcome. Numerous stents grafts must be kept in stock in each narrow diameter range so that the proper size is available, resulting in additional inventory expense.

It would be desirable to have a stent graft sealing system and method that would overcome the above disadvantages.

### SUMMARY OF THE INVENTION

One aspect according to the present invention provides a stent graft having a seal stent, and tubular graft material having a central axis and a pleated seal region. The pleated seal region is supported by the seal stent and urged outward from the central axis by the seal stent. The pleated seal region has an initial deployment diameter and an extended deployment diameter. The extended deployment diameter is greater than one and one-half times the initial deployment diameter.

Another aspect according to the present invention provides a stent graft including tubular graft material having a central axis and a seal region, the tubular graft material having uninterrupted longitudinal pleating; and a seal stent supporting the seal region, the seal stent urging the seal region outward from the central axis.

Another aspect according to the present invention provides a stent graft including tubular graft material having a central axis, a pleated seal region, and an unpleated body region; and a seal stent supporting the seal region, the seal stent urging the pleated seal region away from the central axis.

Another aspect according to the present invention provides a method for sealing a stent graft at a seal zone of a body lumen, including providing a stent graft having a seal stent and a tubular graft material, the tubular graft material having a central axis and a pleated seal region, the pleated seal region being supported by the seal stent and urged away from the central axis by the seal stent, the pleated seal region having an initial deployment diameter and an extended deployment diameter greater than one and one-half times the initial deployment diameter; advancing the stent graft through the body lumen in a compressed state until the pleated seal region aligns with the seal zone; and deploying the stent graft to a relaxed state in the body lumen at the initial deployment diameter.

The foregoing and other features and advantages will become further apparent from the following detailed description, read in conjunction with the accompanying drawings. The detailed description and drawings are merely illustrative.

### BRIEF DESCRIPTION OF THE DRAWINGS

FIGS. 1A-1B are side and detailed perspective views, respectively, of a stent graft in accordance with the present invention;

FIGS. 2A-2C are end views of stent grafts in accordance with the present invention;

FIG. 3 is a side view of another embodiment of a stent graft made in accordance with the present invention;

FIG. 4 is a side view of another embodiment of a stent graft in accordance with the present invention; and

FIG. 5 is a flowchart of a method of sealing a stent graft at a seal zone of a body lumen in accordance with the present invention.

### DETAILED DESCRIPTION

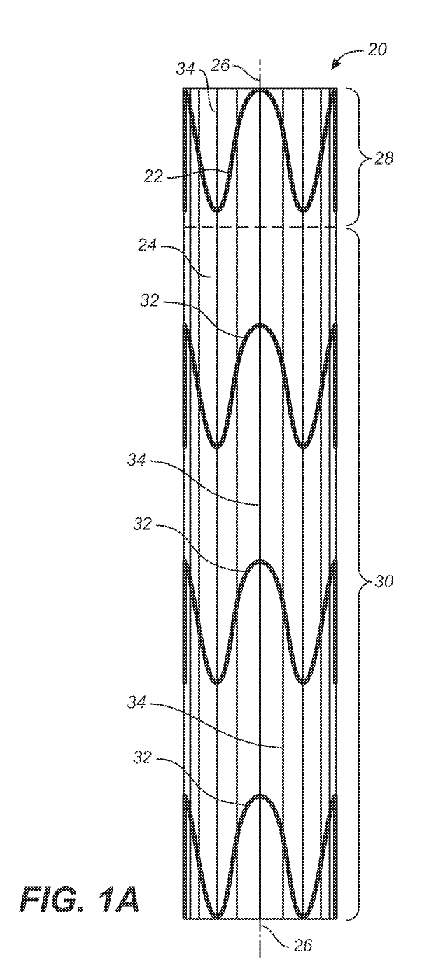

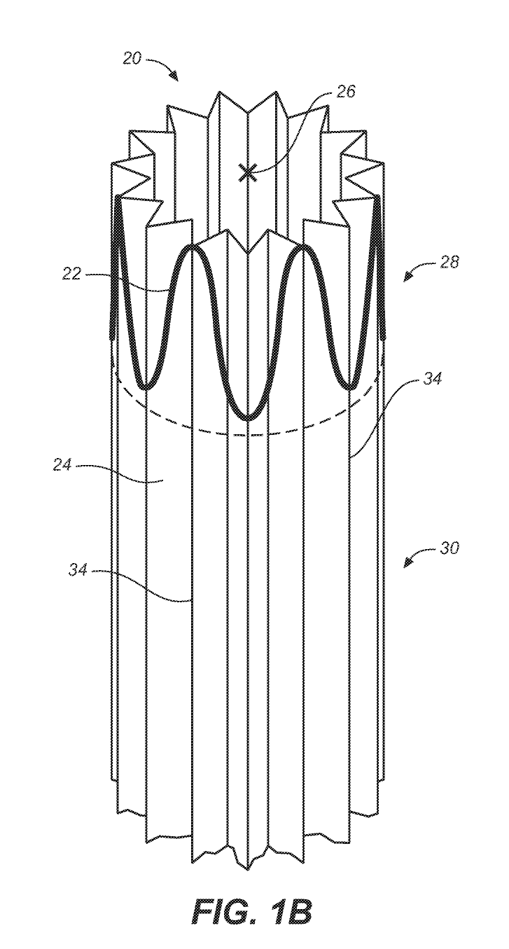

FIGS. 1A-1B, in which like elements share like reference numbers, are side and detailed perspective views, respectively, of a stent graft. The stent graft 20, illustrated in the deployed state, includes a seal stent 22 and tubular graft material 24 with a central axis 26 and a pleated seal region 28. The pleated seal region 28 is supported by the seal stent 22 and urged outward from the central axis 26 by the seal stent 22. The pleated seal region 28 has an initial deployment diameter and an extended deployment diameter. In one embodiment, the extended deployment diameter is greater than one and one-half times the initial deployment diameter. The stent graft 20 also has a body region 30 with at least one body stent 32 supporting the body region 30. In this example, the pleats 34 are uninterrupted longitudinal pleating in a radial pleating pattern, extending continuously across the pleated seal region 28 and the body region 30.

The stent graft 20 is deployed in the body lumen with the pleated seal region 28 at an initial deployment diameter defined as the diameter of the body lumen at the time of initial stent graft deployment. As the body lumen changes diameter, due to growth or otherwise, the pleats 34 allow the diameter of the pleated seal region 28 to change, so that the diameter of the pleated seal region 28 continues to match the changing body lumen diameter. This maintains the seal between the pleated seal region 28 and the body lumen wall at a seal zone of the body lumen wall adjacent the pleated seal region 28. The extended deployment diameter is defined as the largest diameter of the pleated seal region 28 that is possible, i.e., the diameter at which the pleats 34 in the pleated seal region 28 have flattened out and the tubular graft material 24 is fully expanded. The extended deployment diameter is the limit at which the pleated seal region 28 can maintain a seal with the seal zone of the body lumen wall. In one embodiment, the extended deployment diameter is greater than one and one-half times the initial deployment diameter.

The stent graft 20 can be a straight or bifurcated stent graft. The seal stent 22, body stent 32, and the tubular graft material 24 can be any stent and graft material typically used for stent grafts. The seal stent 22 and body stent 32 can be self-expanding and can be a series of individual spring stents as illustrated in FIG. 1A. In another embodiment, the seal stent 22 and/or body stent 32 can be tubular lattice stents, such as lattice stents with diamond-shaped openings in the stent. In another embodiment, the seal stent 22 can be a single flexible unit along the length of the pleated seal region 28 and the body stent 32 can be a single unit along the length of the body region 30. The stents can be made of can be made of spring steel, stainless steel, titanium, nickel titanium alloys (Nitinol), a polymer or copolymer, a combination of these materials, or other suitable super elastic and/or shape memory materials. The stents may include books or barbs to promote fixation and holding. The seal stent 22 urges the pleated seal region 28 outward from the central axis 26 to maintain the seal between the pleated seal region 28 and the body lumen wall. In one embodiment, the seal stent 22 can extend longitudinally beyond the pleated seal region 28 and/or beyond the end of the stent graft 20. Extending beyond the end of the stent graft 20 helps the seal stent 22 fix the stent graft 20 in place by greater contact of the seal stent 22 with the body lumen wall. When the stent graft 20 is used to treat an abdominal aortic aneurysm, the portion of the stent graft 20 extending beyond the stent graft 20 allows blood flow through that portion, such as allowing blood flow to the renal arteries from the abdominal aorta.

The tubular graft material 24 can be any woven or interlocked graft material capable of holding a pleat and suitable for stent grafts, such as woven polymer materials, e.g., Dacron, or polytetrafluoroethylene (PTFE), or interlocked graft materials including knit, stretch, and velour materials. The tubular graft material 24 can be different material in the pleated seal region 28 and the body region 30. The tubular graft material 24 can be formed as a sheet and pleated into the desired pleating pattern, such as a radial pleat, triangular pleat, unidirectional tangential pleat, bidirectional tangential pleat, or other pleating pattern desired, before the tubular graft material 24 is attached to the stents. The tubular graft material 24 can be attached to the seal stent 22 and body stents 32 by sewing and/or adhesive. The attachment is made so the tubular graft material 24 can expand in diameter without binding.

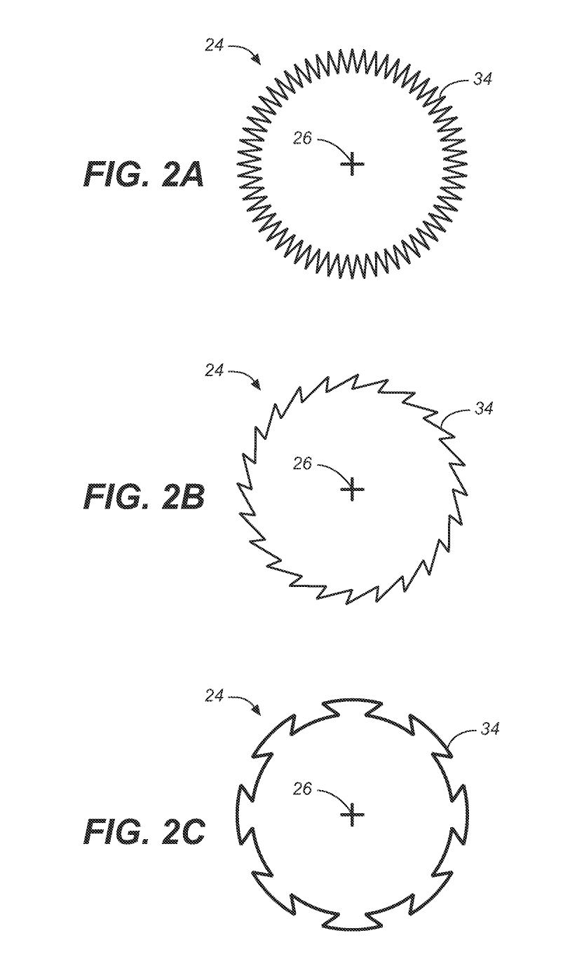

FIGS. 2A-2C, in which like elements share like reference numbers with each other and with FIG. 1, are end views of stent grafts. FIGS. 2A, 2B, and 2C illustrate a radial pleat, unidirectional tangential pleat, and a bidirectional tangential pleat, respectively. The pleating allows the tubular graft material 24 to expand over a wide range of diameters. Fluid pressure from the fluid in the body lumen holds the tubular graft material 24 against the body lumen wall and helps maintain the seal between the stent graft and the body lumen wall.

Referring to FIG. 2A, the pleating pattern of the tubular graft material 24 is a radial pleat including inner folds 40 and outer folds 42, joined by radial surfaces 44. In one embodiment, the tubular graft material 24 can be attached to the seal stent at the outer folds 42. The radial surfaces 44 lie along the radial direction of the tubular graft material 24 when the stent graft is in a compressed state.

Referring to FIG. 2B, the pleating pattern of the tubular graft material 24 is a unidirectional tangential pleat including inner folds 46 and outer folds 48, joined by overlapping tangential surfaces 50. In one embodiment, the tubular graft material 24 can be attached to the seal stent at the outer folds 48. The overlapping tangential surfaces 50 lie tangent to a circle formed by the tubular graft material 24 when the stent graft is in a compressed state. Adjacent overlapping tangential surfaces 50 overlap each other when the stent graft is in a compressed state and can overlap after the stent graft is deployed.

Referring to FIG. 2C, the pleating pattern of the tubular graft material 24 is a bidirectional tangential pleat including inner surfaces 52 and outer surfaces 54, joined by opposed tangential surfaces 56. In one embodiment, the tubular graft material 24 can be attached to the seal stent at the outer surfaces 54. The inner surfaces 52, outer surfaces 54, and opposed tangential surfaces 56 lie tangent to a circle formed by the tubular graft material 24 when the stent graft is in a compressed state. The opposed tangential surfaces 56 overlap the inner surfaces 52 and outer surfaces 54 when the stent graft is in a compressed state and can overlap after the stent graft is deployed.

Those skilled in the art will appreciate that the pleating patterns illustrated in FIGS. 2A-2C are but a few examples of the pleating patterns that can be used with the stent graft. The pleating pattern can be any pleating pattern expandable over a wide range of stent graft diameters. For example, the relative dimensions of the pleating patterns discussed above can be selected to achieve a particular result, such as a small stent graft diameter in the compressed state, stent graft flexibility in the compressed state, and/or stent graft expandability in the relaxed state. Relative dimensions of the radial surfaces 44, overlapping tangential surfaces 50, inner surfaces 52, outer surfaces 54 and/or opposed tangential surfaces 56 can be selected. Varying the relative dimensions varies the frequency and overlap of the pleating pattern.

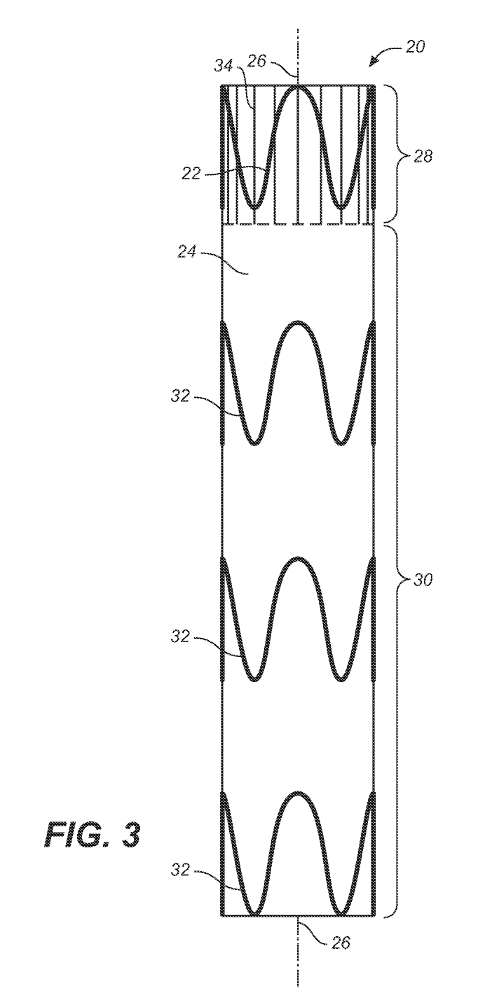

FIG. 3, in which like elements share like reference numbers with each other and with FIG. 1, is a side view of another embodiment of a stent graft. In this example, the tubular graft material 24 has a pleated seal region 28, which is a radial pleat, and an unpleated body region 30. The two regions are joined by sewing or other conventional techniques or by providing a flat sheet with a radially diverging end (cone shape) in a one piece material (not shown). Those skilled in that art will appreciate that the tubular graft material 24 can have the pleated seal region 28 in different longitudinal locations, such as the proximal end, distal end, or middle of the stent graft 20. The tubular graft material 24 can include more than one pleated seal region 28, such as pleated seal regions of the stent graft 20 at both ends, or both ends and the middle. When the stent graft 20 is a bifurcated stent graft, a pleated seal region can be provided at the end of each of the legs.

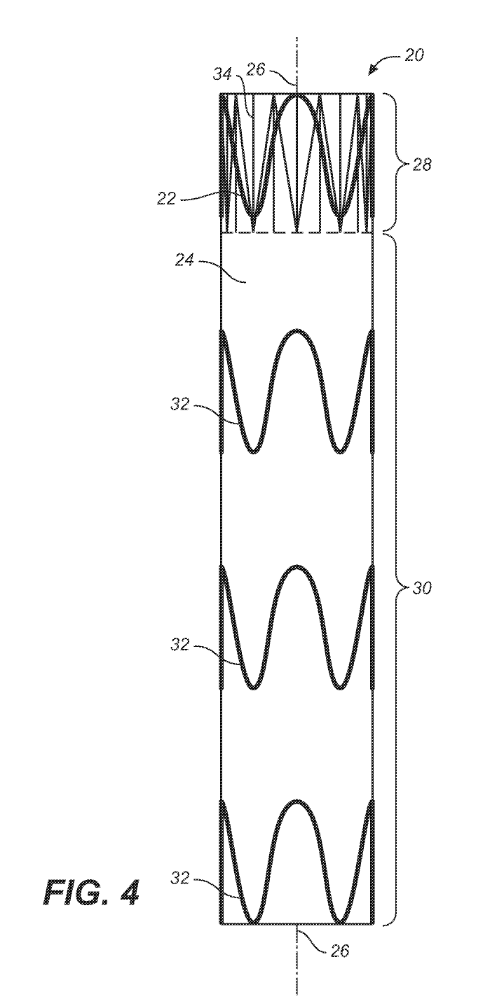

FIG. 4, in which like elements share like reference numbers with each other and with FIG. 1, is a side view of another embodiment of a stent graft. In this example, the tubular graft material 24 has a pleated seal region 28, which is a triangular pleat, and an unpleated body region 30. The triangular pleat provides a smooth transition between the pleated seal region 28 and the unpleated body region 30.

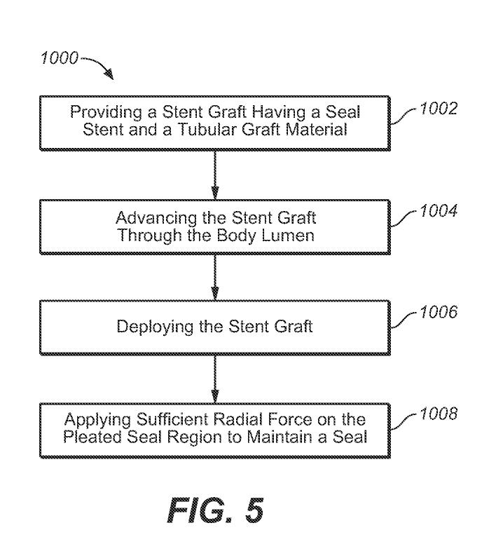

FIG. 5 is a flowchart of a method of sealing a stent graft at a seal zone of a body lumen. The method 1000 includes providing a stent graft having a seal stent and a tubular graft material 1002, advancing the stent graft through the body lumen 1004, and deploying the stent graft 1006. The method 1000 can further include applying sufficient radial force on the pleated seal region to maintain a seal 1008. Exemplary body lumens include the

Providing a stent graft having a seal stent and a tubular graft material 1002 includes providing a stent graft having a seal stent and a tubular graft material, with the tubular graft material having a central axis and a pleated seal region. The pleated seal region is supported by the seal stent and urged away from the central axis by the seal stent. The pleated seal region has an initial deployment diameter, and an extended deployment diameter greater than one and one-half times the initial deployment diameter.

Advancing the stent graft through the body lumen 1004 includes advancing the stent graft through the body lumen in a compressed state until the pleated seal region aligns with the seal zone. The seal zone can be any portion of the body lumen where a seal can be maintained. For treatment of an abdominal aortic aneurysm, the seal zone can be upstream of the aneurysm, such as between the renal arteries and the aneurysm. For treatment of a thoracic aortic transection, the seal zone can be upstream and/or downstream of the transection. The stent graft can be advanced through a catheter containing the stent graft in a compressed state after advancing a guidewire through the body lumen and positioning a catheter over the guidewire.

Deploying the stent graft 1006 includes deploying the stent graft to a relaxed state in the body lumen at the initial deployment diameter. The stent graft relaxes from the compressed state to the relaxed state on exiting the catheter with the pleated seal region contacting the seal zone. The pleated seal region expands to the initial deployment diameter.

Applying sufficient radial force on the pleated seal region to maintain a seal 1008 includes applying sufficient radial force on the pleated seal region with the seal stent to maintain a seal between the pleated seal region and the body lumen for diameters of the body lumen between the initial deployment diameter and the extended deployment diameter. The seal stent urges the pleated seal region against the seal zone of the body lumen, maintaining the seal for diameters of the body lumen up to the extended deployment diameter. The broad range of diameters between the initial deployment diameter and the extended deployment diameter (greater than one and one-half times the initial deployment diameter) allows for growth of the body lumen.

While specific embodiments of the invention are disclosed herein, various changes and modifications can be made without departing from the spirit and scope of the invention.

1. A stent graft comprising:
a seal stent; and
a tubular graft material having a central axis and a pleated seal region, the pleated seal region being supported by the seal stent and urged outward from the central axis by the seal stent, the pleated seal region having an initial deployment diameter and an extended deployment diameter;
wherein the extended deployment diameter is greater than one and one-half times the initial deployment diameter.

2. The stent graft of claim 1 wherein the pleated seal region has a radial pleat.

3. The stent graft of claim 1 wherein the pleated seal region has a pleat selected from the group consisting of a triangular pleat, unidirectional tangential pleat, and a bidirectional tangential pleat.

4. The stent graft of claim 1 wherein the pleated seal region is attached to the seal stent by an attachment selected from the group consisting of sewing and adhesive.

5. The stent graft of claim 1 wherein the seal stent extends longitudinally beyond the pleated seal region.

6. The stent graft of claim 1 wherein the tubular graft material has a body region and further comprising at least one body stent supporting the body region.

7. A stent graft comprising:
a tubular graft material having a central axis and a seal region, the tubular graft material having uninterrupted longitudinal pleating; and
a seal stent supporting the seal region, the seal stent urging the seal region outward from the central axis.

8. The stent graft of claim 7 wherein the uninterrupted longitudinal pleating is a radial pleat.

9. The stent graft of claim 7 wherein the uninterrupted longitudinal pleating is a pleat selected from the group consisting of a unidirectional tangential pleat and a bidirectional tangential pleat.

10. The stent graft of claim 7 wherein the seal region is attached to the seal stent by an attachment selected from the group consisting of sewing and adhesive.

11. The stent graft of claim 7 wherein the seal stent extends longitudinally beyond the seal region.

12. The stent graft of claim 7 wherein the tubular graft material has a body region and further comprising at least one body stent supporting the body region.

13. The stent graft of claim 7 wherein the seal region has an extended deployment diameter greater than one and one-half times an initial deployment diameter.

14. A stent graft comprising:
a tubular graft material having a central axis, a pleated seal region, and an unpleated body region; and
a seal stent supporting the seal region, the seal stent urging the pleated seal region away from the central axis.

15. The stent graft of claim 14 wherein the pleated seal region has a radial pleat.

16. The stent graft of claim 14 wherein the pleated seal region has a pleat selected from the group consisting of a triangular pleat, unidirectional tangential pleat, and a bidirectional tangential pleat.

17. The stent graft of claim 14 wherein the pleated seal region is attached to the seal stent by an attachment selected from the group consisting of sewing and adhesive.

18. The stent graft of claim 14 wherein the seal stent extends longitudinally beyond the pleated seal region.

19. The stent graft of claim 14 further comprising at least one body stent supporting the unpleated body region.

20. The stent graft of claim 14 wherein the pleated seal region has an extended deployment diameter greater than one and one-half times an initial deployment diameter.

21. A method for sealing a stent graft at a seal zone of a body lumen, the method comprising:
providing a stent graft having a seal stent and a tubular graft material, the tubular graft material having a central axis and a pleated seal region, the pleated seal region being supported by the seal stent and urged away from the central axis by the seal stent, the pleated seal region having an initial deployment diameter and an extended deployment diameter greater than one and one-half times the initial deployment diameter;
advancing the stent graft through the body lumen in a compressed state until the pleated seal region aligns with the seal zone; and
deploying the stent graft to a relaxed state in the body lumen at the initial deployment diameter.

22. The method of claim 21 further comprising applying sufficient radial force on the pleated seal region with the seal stent to maintain a seal between the pleated seal region and the body lumen for diameters of the body lumen between the initial deployment diameter and the extended deployment diameter.

23. The method of claim 21 wherein the body lumen is selected from the group consisting of a thoracic artery and an abdominal artery.
